(19) United States
(12) Patent Application Publication
(10) Pub. No.: US 2022/0289869 A1
(43) Pub. Date: Sep. 15, 2022

(54) Purification of Reptilian Hyaluronic Acid and Its Use for Soft and Hard Tissue Repair and Regeneration

(71) Applicant: Lacerta Life Sciences, LLC, Abbeville, LA (US)

(72) Inventors: Mitchell C. Sanders, Southborough, MA (US); Da Wei, Natic, MA (US)

(21) Appl. No.: 17/828,325

(22) Filed: May 31, 2022

Related U.S. Application Data
(63) Continuation of application No. PCT/US20/62563, filed on Nov. 30, 2020.
(60) Provisional application No. 62/942,104, filed on Nov. 30, 2019.

(51) Int. Cl. C08B 37/08; A61L 27/20; A61L 27/36; A61L 15/28

(52) U.S. Cl. CPC C08B 37/0072; A61L 27/20; A61L 27/3604; A61L 15/28; A61L 2430/02; A61L 2430/34; A61L 2300/412

(57) ABSTRACT

An ultra-rapid method for the isolation and purification of high molecular weight hyaluronic acid (HA) from the skin of reptiles (rHA). The method utilizes an extraction buffer that inhibits unwanted hyaluronidases, filtration to remove cell debris and precipitated proteins, and a highly substituted anion exchange column at a low pH with a high salt elution to maximize the yield and purity of rHA per kilogram of skin. rHA is extremely charged and elutes between 1.6-3M NaCl. Our findings suggest that rHA is high molecular weight (>2 mDa) and has a profound ability to enhance cell migration and proliferation for wound repair and regeneration. Preliminary evidence suggests that scaffolds of rHA are ideal for biomaterial engineering that could be incorporated into several medical devices and pharmaceutical products. We have identified novel crosslinking methods to streamline esterification of rHA for advanced wound care products as well as novel conjugates to improve the osteogenic and osteoconductive potential of rHA for bone regeneration and repair.

### CROSS-REFERENCE TO RELATED APPLICATIONS

This application claims priority of International Application PCT/US2020/062563 filed on Nov. 30, 2020, which itself claimed priority of Provisional Patent Application 62/942,104 filed on Nov. 30, 2019. The disclosures of both prior applications are hereby incorporated herein by reference for all purposes.

### BACKGROUND

This application relates to the purification of reptilian hyaluronic acid, and uses of the purified substance.

Hyaluronic Acid (HA) is a non-sulfated glycosaminoglycan which is composed of alternating D-glucuronic acid and N-acetyl-D-glucosamine sugars, β-(1 to 4) and β-(1 to 3) glycosidic bonds. HA is an essential extracellular matrix (ECM) component of vertebrates that is a ubiquitous ECM component and abundant in the skin, vitreous humor, umbilical cord, and synovial fluid. Most commercial sources of hyaluronic acid are purified from rooster comb or synthesized in avirulent strains of Streptococcus sp. Purification methods include but are not limited to treatment with proteolytic enzymes, organic solvents, cationic quaternary ammonium compounds and detergents for precipitation. HA from rooster comb is typically purified in 15-25 steps which includes common methods of tissue isolation, grinding, heat inactivation, extraction, protease treatment (papain, pepsin, actinase E), precipitation with cetylpyridinium chloride (CPC), dialysis, and lyophilization.

HA has been purified from fermentation media of Streptococcus sp. Recent improvements in the purification have established that DNA, protein and endotoxin impurities can be removed by ultrafiltration or centrifugation using a combination of low pH (2.0) with trichloroacetic acid (TCA) and 1-2% charcoal and gamma alumina to remove endotoxin impurities.

Most animal-based HA is not practical for commercialization due to the numerous steps, low yield, high impurities and high production costs. The skin of reptiles is very challenging for the purifying of HA, at least in part because the epidermis is heavily keratinized and the dermis is extremely rich in connective tissue and pigmentary cells. Previous approaches to purify and characterize reptile HA (rHA) in other labs has been unsuccessful because the HA is high molecular weight and extremely charged. The high molecular weight rHA approaches 2 million Daltons (mDa), as such, the material has a very high viscosity which makes it challenging to purify by conventional methods such as proteolysis, heat inactivation ultrafiltration, CPC or TCA precipitation, and centrifugation. By example, proteolysis is used to remove proteins from the rooster comb or bacterial preparations, however, proteolysis of rHA fractions from reptilian skin results in the release of black melanin which irreversibly binds to the HA throughout the purification. Heat inactivation is normally a great step in the purification of HA from non-reptilian sources because it inactivates bacterial and host hyaluronidases. However, in the case of reptilian skin, which is rich in collagen and keratin, after heating, the preparation becomes a solid block of gelatin, which makes it impossible to separate the ECM proteins from the rHA. Furthermore, CPC and TCA precipitation of HA in reptilian skin are fraught with protein, nucleic acid, and endotoxin contaminants due to the strongly anionic charge of the rHA. Lastly, reptile skin is rich in bacterial & reptile hyaluronidases and metal ion impurities which make it ultrasensitive to enzymatic and oxidative degradation which further makes the purification challenging.

### SUMMARY

Aspects of this disclosure relate to a rapid and simple method to purify HA from reptile which provides for the high yield, purity and low cost of production of highly charged and high molecular weight HA from alligator and other reptiles (rHA).

All examples and features mentioned below can be combined in any technically possible way.

In one aspect, this disclosure features a method for purifying reptilian hyaluronic acid (rHA).

Some examples include one of the above and/or below features, or any combination thereof. In some examples the rHA is developed from reptilian skin that is ground to a 1-6 mm particle size and then extracted overnight in a buffer that inactivates hyaluronidases. In an example the buffer is at a pH of 8.0-8.5 and is composed of 10-100 mM Tris EDTA, 0.01-1% SDS, and 0.1-1 mM EDTA. In some examples the extract is filtered in multiple steps at progressively finer filter pore sizes from multiple micron to sub-micron sizes. to remove the unextracted material. In some examples the filtered extract is titrated to pH 4.5-5.5 with hydrochloric acid (HCL) or acetic acid (CH₃COOH), then purified by anion exchange chromatography, and the eluted with NaCl. In an example the rHA elutes from 1.5-2M NaCl with >98% yield and at least 99% purity. In an example the rHA is high molecular weight≥2 mDa and is extremely charged and hydrophilic. In an example the strongly anionic and hydrophilic rHA is used as a carrier for drug delivery.

Some examples include one of the above and/or below features, or any combination thereof. In some examples the purified rHA is used for hard tissue repair or soft tissue repair. In an example the soft tissue repair is for the healing of chronic and acute wounds. In an example the rHA is chemically modified by crosslinking with L-serine benzyl ester (SBE) with EDC (N-(3-dimethylaminopropyl)-N′-ethylcarbodiamide, NHS (N-hydroxysuccimide) or a combination thereof. In an example the crosslinked SBE-rHA or rHA is aerated or whipped into a foam and then dried or freeze dried into a sheet. In an example the SBE-rHA is lined with silicone, polyvinyl alcohol (PVA) or polyethylene to form a biocomposite advanced wound dressing.

In an example the hard tissue repair is the fusion or healing of bones or cartilage. In an example the rHA is made osteo-inductive and/or osteoconductive through the cross-linking of rHA with carboxymethyl cellulose (CMC), collagen, bone morphogenic proteins (BMPs), titanium dioxide, halloysite nanotubes (HNTs), bioactive borosilicate, polysorbitol sebacate glutamate (PSSG), polycaprolactone, or polyvinyl alcohol. In an example the rHA-osteogenic material is mixed with autologous demineralized bone or hydroxyapatite for the treatment of patients requiring spinal fusion. In an example the rHA-osteogenic material is mixed with mesenchymal stem cells to assist in the differentiation and repair of the damaged bone or cartilage material. In an example the rHA-osteogenic material is used to treat bone defects, including but not limited to spinal fusion or repair, surgically created osseous defects, or osseous defects created from traumatic injury.

In another aspect this disclosure features purified reptilian HA (rHA) derived from reptilian skin.

Some examples include one of the above and/or below features, or any combination thereof. In an example the rHA has a molecular weight of at least 2 million Daltons. In an example the rHA is highly charged and hydrophilic. In some examples the reptilian skin is derived from alligators, crocodiles, caimans, and/or gharial.

### BRIEF DESCRIPTION OF THE DRAWINGS

FIG. 1 is a chromatograph of anion exchange purification of rHA.

FIG. 2 illustrates the optical density of samples of rHA and bacterial HA (bHA).

FIG. 3 illustrate the absorbance of rHA compared to that of bHA and rooster comb HA (rcHA).

FIG. 4 provides results of cell proliferation assays with rHA, bHA, and rooster comb HA (RC).

FIG. 5 is a schematic diagram of an rHA purification process.

### DETAILED DESCRIPTION

Here we report a novel, rapid (1 day), and simple (5 steps) purification of rHA involving the following steps: (1) grinding, (2) extraction, (3) filtration, (4) anion exchange purification, and 5) tangential flow filtration. The rHA can be derived from the skin of different reptile species, including but not limited to alligators, crocodiles, caimans, and/or gharial.

### Tissue Grinding

Based on several grinding ball mills and emulsifier methods, we have determined that the frozen skin from reptiles can be finely ground to an approximately 1-6 mm particle size by the use of several commercially available grinding machines, with the preferred approach being an industrial impact grinder. We utilized three different methods to optimize the release of reptilian hyaluronic acid (rHA) from skin. Based on HA extraction from the ground material we determined an impact grinder had the highest yield, while the mortar grinder had the highest molecular weight rHA. 1% agarose gel stained with 0.005% stains will show the molecular weight distribution of rHA derived from the American alligator skin using the three different grind methods: 1) mortar grinder, 2) impact grinder, and 3) impeller grinder. The mortar grinder method gave the highest molecular weight distribution of rHA suggesting that it is more gentle than the impact and impeller grinders. A mortar grinder is recommended for highly viscous powders, suspensions and pastes. In contrast the impact grinder and impeller may be better suited for the grinding of hard and brittle materials.

Table 1 presents results using three different grinders.

TABLE 1

| | 1)Mortar | 2)Impact | 3)Impeller |
|---|---|---|---|
| | Grinder | Grinder | Grinder |
| HA Released/ | 148.47 μg | 159.94 μg | 155.45 μg |
| g tissue | | | |
| MW based on | High | Medium | Medium |
| 1% Agarose Gel | | | |

The material should be kept as cool as possible (preferably in the range −80° C.-4° C.) to prevent the connective tissue from gumming up and clogging the grinder machine. We also determined that higher temperatures may also reduce the high molecular weight (HMW) of the rHA due to the oxidative and hyaluronidase degradation, described above. The optimal particle size is about 1-6 mm in order to get the most efficient extraction of HA from the tissue. Ground material measured using the Verder method provides a uniform 1-2 mm particle size based on phase contrast microscopy at 10×.

### Extraction

We devised an extraction buffer with ingredients that were demonstrated to inhibit both reptilian and bacterial hyaluronidases. The extraction conditions were also optimized to prevent shearing of the HMW rHA. In the preferred embodiment, the finely ground material is extracted at 4° C. without shaking in a buffer containing Tris-EDTA-SDS (10-50 mM Tris, 0.1-10 mM EDTA, and 0.01-0.5% SDS). Higher temperatures and/or mechanical ball shaking results in non-ideal degradation of rHA. The extraction time is overnight (˜16 hours), but a second extraction can be performed to isolate 25% more rHA per preparation.

### Filtration

The overnight extract of HA is reduced to pH 5.5-6.2 with dropwise addition of HCL or acetic acid while stirring and then sequentially filtered (2% Celite, 10 μm glass fiber, 1 μm, 0.45 μm, and 0.2 μm polyether sulfone membranes) prior to anion exchange chromatography. For industrial applications we have determined that the material can be filtered by a sequential pore size depth filtration. We determined the best depth filtration methods to purify rHA include the sequential use of depth filters to remove unwanted proteins prior to anion exchange chromatography. The best combination of filters that were investigated includes a 4-9 μm depth filter followed by a 3-6 μm depth filter and then two 0.2-5 μm depth filters prior to sterile filtering at 0.2 μm. Lower pH than 5.5 results in uncontrolled protein precipitation and clogging of the filtration membranes. We use a low pH to reduce the binding of protein impurities to a highly substituted weak anion exchange column (STIC PA, Sartorius, Gottingen, Germany).

### Purification

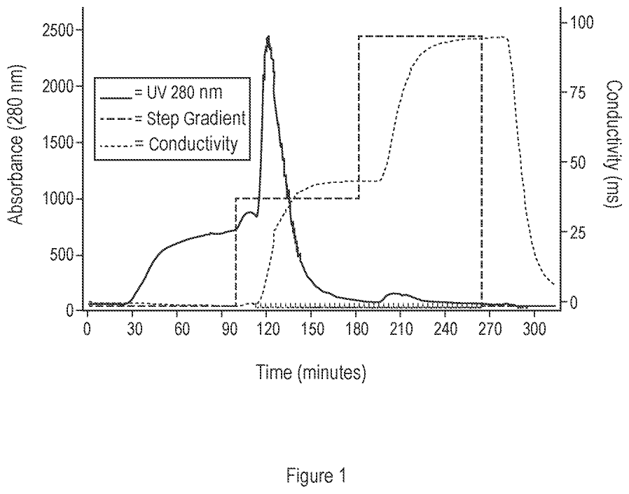

The 1 ml column can be loaded at a high flow rate of 0.02-1.5 L/min and the residual protein can be eluted at 0.6-0.8 M NaCl. For scale 28-8 L STIC cassettes (24.2 L) run at a flow at 1.5-5 L/min. The sample is equilibrated with a buffer that reduces the binding of proteins during the load and rinse of the sample (Buffer A 20-50 mM Sodium Acetate pH 5.5, 5 mM EDTA). Proteins that bind to the STIC PA resin are removed by rinsing with 4 membrane volumes containing 0.6-0.8M salt (Buffer B 20-50 mM sodium acetate, 5 mM EDTA, and 06-0.8M NaCl). The HA is eluted with a high salt and pH buffer (C Buffer: 20 mM Tris pH 8.5, 2-3M NaCl). For optimal yield, the rHA is eluted at a slower flow rate 0.5-1 L/min. We determined that rooster comb and Streptococcal HA elutes at 0.5 M NaCl on a STIC PA anion exchange column, while rHA elutes at 1.8-3 M NaCl suggesting that it is much more anionic than other reported HA species. The chromatograph of the STIC PA column is shown in FIG. 1 for a 75 ml STIC PA cassette loaded with a 500 ml overnight extract of reptilian skin. The material loaded at pH 5.5 was washed with 0.8 N NaCl to remove protein and nucleic acid impurities and then the rHA was eluted from the column with a 2M step gradient of NaCl. Our findings indicate that rHA can be purified by a novel salt tolerant anion exchange chromatography (STIC PA). Our initial attempts to use classical anion resins (DEAE and QAE) were unsuccessful. The weak anion exchange resin DEAE did not separate the rHA from the protein contaminants, The strong quaternary amine resin (QAE) eluted the proteins but we were unable to get the HA off the column even in 2M NaCl.

In Table 2 the yield and purity of HA are set forth. Based on HA determination (Purple-Jelly Kit, Biocolor), protein, nucleic acid and endotoxin assays, the purified rHA is extremely pure and has <1-2% protein, nucleic acid and endotoxin impurities, with repeatable results.

TABLE 2

| | STIC PA | STIC PA | STIC PA |
|---|---|---|---|
| | (Run 1) | (Run 2) | (Run 3) |
| rHA (mg/ml) | 10.92 | 3.50 | 2.94 |
| Protein (mg/ml) | 0.0687 | 0.01717 | 0.02827 |
| Nucleic Acid | 0.00204 | 0.00299 | 0.00089 |
| (mg/ml) | | | |
| Endotoxin | 0.0854 | 0.2060 | 0.2219 |
| (EU/ml) | | | |
| Purity (%) | 99.43 | 99.43 | 99.02 |

### Tangential Flow Filtration

For analytical methods the rHA can be concentrated and desalted using a 100 k molecular weight cut off (MWCO) Centriprep spin filter. Following concentration, the rHA is recentrifuged 3× with deionized water to desalt the ultrapure rHA sample as described by the manufacturer (Millipore Sigma, Burlington Mass.). For commercial scale up, we utilized two fully integrated KrosFlo© KMPi TFF Systems to perform the same process. Briefly, The high salt elution of the rHA from the STIC Cassettes, was concentrated 30× to 1-2 mg/ml, desalted with three diavolumes of deionized and endotoxin free water to a final salt concentration of 150 mM NaCl, and sequentially size selected using two tangential flow 100 k and 10K MWCO hollow fiber membranes in series (KF 20 cm i00 kD mPES 0.5 mm and KF 65 cm 10 kD mPES 0.5 mm respectively, Repligen, Boston Mass.). Following overnight desalting the residual endotoxins were removed by incubating with 2% alumina oxide.

We typically think of HA as being composed of equal and alternating D-glucuronic acid and N-acetyl-D-glucosamine. We hypothesize that reptiles have found a way to either synthesize HA with a higher composition D-glucuronic acid, thereby, making it more anionic. Alternatively, the rHA is chemically modified in the ECM by a metal catalyzed oxidation of the N-acetyl-D-glucosamine hydroxyl groups to additional carboxylic acid groups.

Most mammalian HA is colorless, whereas our purified rHA has a brown hue suggesting that it is complexed with metal ions. This finding is consistent with a previous report that suggests that HA can be complexed and stabilized with other metal ions including zinc II and Iron at low pH. To reduce the metal tightly bound to the rHA, we utilized 5 mM EDTA in the chromatography running buffers to reduce the metals bound to the purified rHA. Even after EDTA treatment the purified rHA ha a slightly brownish color at 1-2 mg/ml.

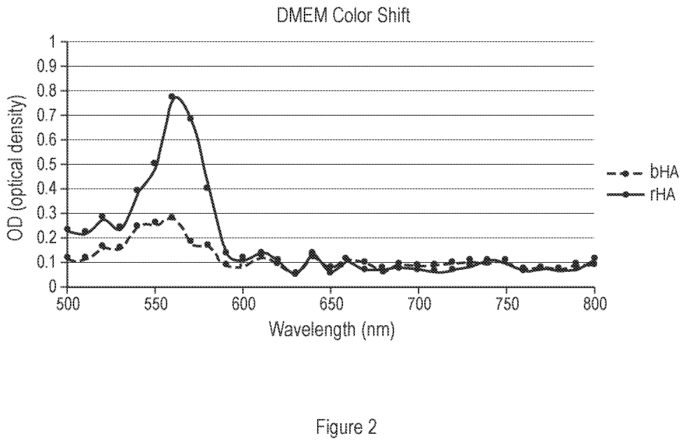

In either case, it is very clear that rHA is more charged (anionic) than HA isolated from mammals and this may confer enhanced metal binding complexes (Metal-HA). To demonstrate this anionic charge of rHA we put it in DMEM media which contains phenol red. Results are shown in FIG. 2. Phenol red is a pH indicator. At low pH (6.0) it is more yellow and at more neutral pH (7) it is more red. rHA is more yellow-red than bacterial HA (bHA) which is red purple. We used phenol red in DMEM media to demonstrate that rHA is much more negatively charged than other mammalian and bacterial derived HA preparations. rHA at 5 mg/ml in water turns phenol red much more yellow-red than 5 mg/ml bacterial derived HA suggesting that it is more acidic than conventional HA from bacteria or rooster comb.

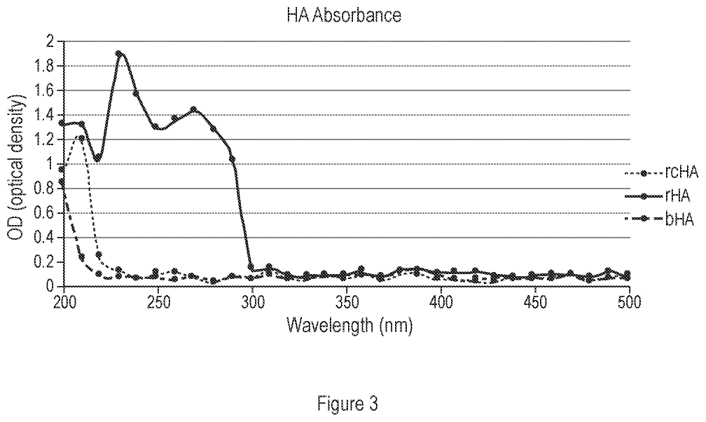

In FIG. 3 we show the absorbance of rHA compared to bacterial (bHA) or rooster comb HA (rcHA). Purified HA from bacteria or rooster comb is free of metal ions. Based on the absorbance spectrum we believe that the metal ion bound is likely Iron (II) or Zinc(II). Reptile HA (rHA) has a broad absorbance peak (three peaks: 190, 240, and 270 nm) consistent with it being bound metal ions (likely iron, Fe²⁺ and Fe³⁺ based on absorbance) whereas, bHA and rcHA have a single peak maxima at 190 nm. Based on protein, nucleic acid and endotoxin assays the eluted rHA from the STIC PA column is ultrapure >98% and the yield from the three step purification method is >>70% yield. We purified rHA from this STIC PA column on an analytical scale (1 ml) and have recently validated with a 75 ml column and a 24.2 L STIC PA cassette system that this one step anion exchange chromatography purification is scalable. The scaled up process is run on an Allegro Pilot Scale chromatography system configured with 28×0.8 L STIC PA cassettes for commercial production of rHA. We have been using this ultrapure HA for the repair and regeneration of soft tissue (skin and wounds) and hard tissue (bones and cartilage). Potential commercial markets for this ultrapure HA include wound care, orthopedics, cosmetics, pharmaceuticals, and consumer health care products, Specific examples include rHA for an advanced wound matrix, combination with a human amnion amnion/chorion matrices (HACM) for preservation of the tissue, complexed with demineralized bone to form a putty that reduces pain for spinal fusions, a wrap or sheath for tendon and nerve repair. An injectable for visco-supplementation of joints (primarily knees and hips), protection as an adhesion barriers, and numerous cosmetic applications (antiaging, antiwrinkle, and anti-scarring creams).

### Cell Proliferation

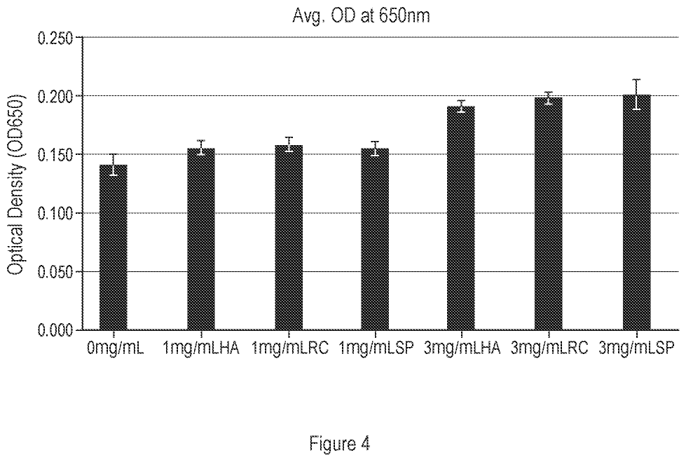

We have determined that rHA due to its high molecular weight (HMW) and strong anionic charge can induce the cell proliferation of Swiss 3T3 fibroblasts through a polyether sulfone membrane suggesting that it would be ideal for inducing the healing of chronic and acute wounds through cell proliferation. rHA may also be important in the healing and fusion of bone material due the HMW and strong anionic charge and metal rHA complexes. Evidence set forth in FIG. 4 suggests that rHA can induce cell proliferation of Swiss 3T3 fibroblasts through a poly ether sulfone membrane in a concentration dependent manner. Similar results were obtained with bacterial (bHA) and rooster comb (RC) HA (+ controls).

### Chemical Crosslinking of rHA

We have chemically crosslinked HA with a serine benzyl ester using the zero length crosslinker (EDC) and NHS. Briefly a 1-2 mg/ml solution of HA is mixed with a 1-5 fold molar excess of EDC (N-(3-dimethylaminopropyl)-N′-ethyl carbodiamide and/or NHS (N-hydroxysuccimide) and the solution is incubated at 1-30 minutes prior to the addition of serine benzyl ester (sBenz-rHA). The crosslinking is allowed to continue for 20-60 minutes and then the free reactive groups are blocked with ethanolamine, alanine, or serine. In some embodiments the sBenz-rHA is frozen at −20° C. and lyophilized for 18-32 hours. In other embodiments, during the 1-60 minute crosslinking, the sample is mixed vigorously in a whisk blender to form a foam and then the material is dried overnight at 70-90° F. with 20-40% relative humidity (RH). In a tube of purified 1.5 mg/ml rHA cross-linked with serine benzyl ester with EDC for 1 hour at room temperature, after the 1 hour incubation the HA-serine benzyl ester forms a cross-linked precipitate, which is visible by phase contrast microscopy at 40×.

In yet another embodiment 200 ml of 1-2 mg/ml rHA is briefly (15 seconds) mixed on a low setting with 20 ml of 0.5M MES buffer pH 5.5, 1.0 ml of EDC, 292 mg of serine benzyl ester, and 292 mg of NHS. After 1 minute of incubation the reaction is mixed on high for 3-4 minutes to form a foam matrix. The foam matrix is poured into parchment paper at a thickness of ⅝ to 12 inch sheets and then dried overnight at 70-90° F. with 20-40RH. After overnight drying the advance rHA matrix can be cut into various sizes and pouched prior to gamma sterilization (25-35 (kGy)). It should be noted that the speed of mixing is important, and that a whisk attachment can be used to whip up the foam matrix.

Crosslinking rHA with Osteoconductive and Osteogenic Material

In another embodiment we have crosslinked rHA with carboxymethylcellulose (CMC) using a similar approach used to form plant derived CMC hydrogels. Briefly, sterile filtered HA and CMC solutions were mixed together at a 1:1 ratio in Dulbecco's phosphate-buffered saline (DPBS) at 4° C. containing 10 mM ammonium persulfate and 10 mM ascorbic acid or citric acid. Once evenly mixed, the solution is then combined with hydroxyapatite or demineralized bone (DMB) powder with a final concentration of CMC-rHA at 3-5% and DMB at 30-40%. The material is then filled into sterile syringes that are then packaged and terminally sterilized by gamma irradiation (25-35 kGy). Slight modifications include dissolving the CMC in 1M ascorbic or citric acid prior to adding it to the HA in DMEM and then adding the ammonium persulfate to begin the reaction. In a second approach the sterile CMC and rHA are crosslinked in DPBS containing 1-5 mM amino acid (preferably cysteine), in a two-step reaction. Cysteine is first coupled to rHA using a 1-5 molar excess of EDC, NHS, or EDC:NHS as described above. The hydroxyl groups CMC is coupled with a sulfhydryl-to-hydroxyl crosslinker with an aromatic spacer (PMPI, p-maleimidophenyl isocyanate). After conjugating CMC with PMPI Isocyanate reacts with non-aqueous hydroxyl groups to form carbamate linkage, the CYS-rHA is then added to form a stable thioether linkage. The material is mixed with DMB or hydroxyapatite to form a putty which is syringe filled and gamma irradiated for sterilization.

This approach of crosslinking rHA is not limited to CMC and could also be performed with other osteoconductive materials including but not limited to collagen, bone morphogenic proteins (BMPs), titanium dioxide, halloysite nanotubes (HNTs), bioactive borosilicate, poly (sorbitol sebacate glutamate) (PSSG), polycaprolactone, polyethylene oxide (PEO), and polyvinyl alcohol. The conjugation of rHA with carboxy methyl cellulose can be mixed with hydroxyapatite in a glass syringe to form an osteo inductive spine product. rHA was conjugated to CMC using ammonium persulfate and citric acid for 30 minutes. Following crosslinking the rHA-CMC was mixed with Hydroxyapatite to form an osteoconductive putty for spine regeneration and repair.

### CONCLUSIONS

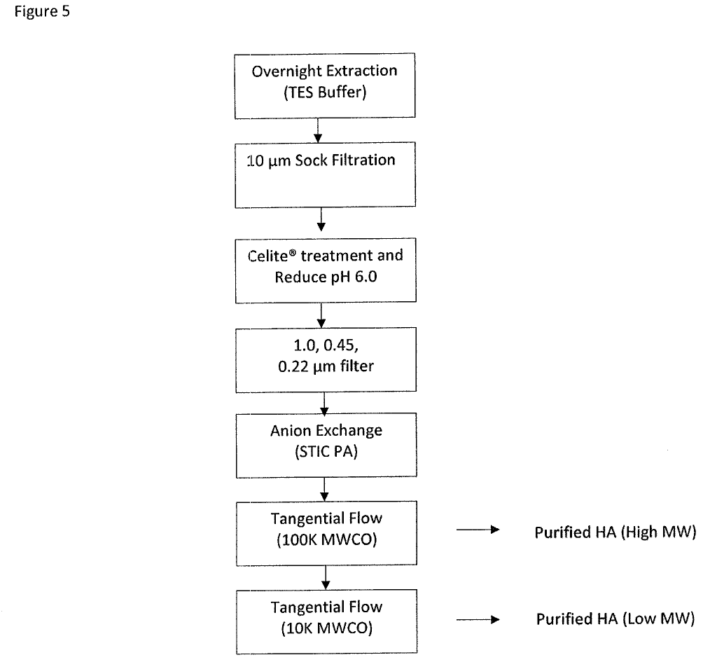

Our novel findings indicate that reptilian HA (rHA) is quite unique in that it is strongly anionic and is bound by metal ions. The strongly anionic rHA maybe an ideal carrier for cell tissue products as well as hydrophilic carrier for drug delivery. Here we report an ultra-rapid (1 day) purification method with very few steps (five steps in some examples). The scaleup schematic of the rHA purification is shown in FIG. 5. We utilized an Allegro Chromatography system configured with 28-0.8 L cassettes of STIC PA. The process is similar to the 1 ml membrane process but is scaled up 22.4 L of membrane with a 150-500 L overnight extraction. The process is semi-continuous in that as soon as the HA elutes from the STIC PA column it is concentrated over a 100K MWCO TFF system. Once the filtrate elutes from the first 100K TFF, the low molecular weight rHA is then concentrated on a 10K MWCO TFF and desalted.

The rHA can be conjugated with serine benzyl ester to form a foam that can be the base layer of an advanced wound dressing for soft tissue repair. We have also developed a novel osteoconductive material based on the conjugation of rHA with carboxy methyl cellulose combined with hydroxyapatite or demineralized bone to form a novel putty for hard tissue (bone and cartilage) repair.

A number of implementations have been described. Nevertheless, it will be understood that additional modifications may be made without departing from the scope of the inventive concepts described herein, and, accordingly, other examples are within the scope of the following claims.

What is claimed is:

1. A method for purifying reptilian hyaluronic acid (rHA).

2. The method of claim 1 wherein the rHA is developed from reptilian skin that is ground to a 1-6 mm particle size and then extracted overnight in a buffer that inactivates hyaluronidases.

3. The method of claim 2 wherein the buffer is at a pH of 8.0-8.5 and is composed of 10-100 mM Tris EDTA, 0.01-1% SDS, and 0.1-1 mM EDTA.

4. The method of claim 2 wherein the extract is filtered in multiple steps at progressively finer filter pore sizes from multiple micron to sub-micron sizes. to remove the unextracted material.

5. The method of claim 3 wherein the filtered extract is titrated to pH 4.5-5.5 with hydrochloric acid (HCL) or acetic acid (CH₃COOH), then purified by anion exchange chromatography, and the eluted with NaCl.

6. The method of claim 5 wherein the rHA elutes from 1.5-2M NaCl with >98% yield and at least 99% purity.

7. The method of claim 6 wherein the rHA is high molecular weight≥2 mDa and is extremely charged and hydrophilic.

8. The method of claim 1 wherein the purified rHA is used for hard tissue repair or soft tissue repair.

9. The method of claim 8 wherein the soft tissue repair is for the healing of chronic and acute wounds.

10. The method of claim 8 wherein the rHA is chemically modified by crosslinking with L-serine benzyl ester (SBE), with EDC (N-(3-dimethylaminopropyl)-N′-ethylcarbodiamide, NHS (N-hydroxysuccimide), or a combination thereof.

11. The method of claim 10 wherein the crosslinked SBE-rHA or rHA is aerated or whipped into a foam and then dried or freeze dried into a sheet.

12. The method of claim 10 wherein the SBE-rHA is lined with silicone, polyvinyl alcohol (PVA) or polyethylene to form a biocomposite advanced wound dressing.

13. The method of claim 8 wherein the hard tissue repair is the fusion or healing of bones or cartilage.

14. The method of claim 13 wherein the rHA is made osteo-inductive and/or osteoconductive through the cross-linking of rHA with carboxymethyl cellulose (CMC), collagen, bone morphogenic proteins (BMPs), titanium dioxide, halloysite nanotubes (HNTs), bioactive borosilicate, polysorbitol sebacate glutamate (PSSG), polycaprolactone, or polyvinyl alcohol.

15. The method of claim 14, wherein the rHA-osteogenic material is mixed with autologous demineralized bone or hydroxyapatite for the treatment of patients requiring spinal fusion.

16. The method of claim 14, wherein the rHA-osteogenic material is mixed with mesenchymal stem cells to assist in the differentiation and repair of the damaged bone or cartilage material.

17. The method of claim 14, wherein the rHA-osteogenic material is used to treat bone defects, including but not limited to spinal fusion or repair, surgically created osseous defects, or osseous defects created from traumatic injury.

18. The method of claim 1 wherein the strongly anionic and hydrophilic rHA is used as a carrier for drug delivery.

19. Purified reptilian HA (rHA) derived from reptilian skin.

20. The rHA of claim 19 having a molecular weight of at least 2 million Daltons.

21. The rHA of claim 19 that is highly charged and hydrophilic.

22. The rHA of claim 19 where the reptilian skin is derived from alligators, crocodiles, caimans, and/or gharial.
